(19) United States
(12) Patent Application Publication
(10) Pub. No.: US 2005/0033106 A1
(43) Pub. Date: Feb. 10, 2005

(54) Synthesis of tris N-alkylated 1,4,7,10-tetraazacyclododecanes

(76) Inventors: Wing Tak Wong, Hong Kong (HK); Cong Li, Changsha (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/885,140

(22) Filed: Jul. 7, 2004

Related U.S. Application Data
(60) Provisional application No. 60/485,219, filed on Jul. 8, 2003.

(51) Int. Cl. A61N 5/02; A61B 5/05

(52) U.S. Cl. 600/2; 600/407

(57) ABSTRACT

A directly synthetic method for preparing tris-alkylated 1,4,7,10-tetraazacyclododecanes by the reactions of 1,4,7,10-tetraazacyclododecane (cyclen) and appropriate electrophiles is accomplished in high yield. The method provides operational convenience, starting material availability, cost economy, atom efficiency and reaction insensitivity to temperature, moisture, and concentrations of starting materials. With this method, the yield of tris-(tert-butoxycarbonylmethyl)-1,4,7,10-tetraazacyclododecane can be 77%, the highest reported. The yield of other tris-N alkylated products can be in the range of 65-84%.

### BACKGROUND OF THE INVENTION

Magnetic Resonance Imaging (MRI) is a well-established and powerful technique for studying the internal structure of the human body, now used in all major hospitals throughout the world. It provides physicians with clear pictures of the interior of the human body from any angle without using hazardous radiation. Compared with other diagnostic methods such as ultrasonography and computerized X-ray torriography (CT), MRI not only excels as a non-invasive method for the three-dimensional imaging of soft tissues in living systems, but also reveals the functional or physiological state of the biological processes of internal organs.

The rapid expansion of medical MRI has prompted the development of contrast-enhancing agents (CAs) which are designed to either enhance the contrast between normal and diseased tissue and/or improve the diagnostic sensitivity and specificity by accelerating the relaxation of water protons in the surrounding tissue. More than 35% of all MRI investigations use a contrast agent, especially in detecting lesions in cancer patients. Due to their advantages, such as unique seven unpaired electrons, very high magnetic moment (μ²=63 μ_{B}²), and symmetric electronic ground state, gadolinium chelates constitute the largest group of MRI contrast agents. Currently, of the seven clinically approved gadolinlum (III) CAs, [Gd(DTPA)(H₂O)]²⁻ (Magnevis™), [Gd(DTPA-BMA)(H₂O)] (Omniscan™), [Gd(BOPTA)(H₂O)]²⁻ (MultiHance™), and [Gd(DTPA-BMEA)(H₂O)] (OptiMARK™) are complexes of acyclic ligands, and three, [Gd(DOTA)(H₂O)] (Dotarem™), [Gd(HP-DO3A)(H₂O)] (ProHance™), and [Gd(DO3A-butrol)(H₂O)] (Gadovist™), are chelated by polyazamacrocycles. All of these Gd³⁺ based chelates share the common characteristics that their ligands occupy eight binding sites at the nine-coordinated metal center and the remaining one coordination site is occupied by a water molecule (hydration number q=1). Compared with acyclic lanthanide chelates, macrocyclic ligands show higher thermodynamic and kinetic stability. For example, 1,4,7,10-tetra(carboxymethyl)-1,4,7,10-tetraazacyclododecane (DOTA) is one of the strongest chelators for Gd(III), being about three orders of magnitude more efficient in complexation efficacy than the best of the previously known chelators, DTPA. Gd-DOTA is also exceedingly stable and inert at physiological pH and in blood serum.

However, the drawbacks of the CAs in current clinical use are well known. First, they are not really organ-specific, and are simply distributed throughout the body via the blood stream. Usually, a dose level of 0.1 mmol/kg of body weight is needed by intravenous injection before the MRI procedure. That means for an adult, several grams of CAs should be used. Second, CAs such as Gd-DTPA and Gd-DOTA are in the form of salts under physiological conditions because of their overall negative charge, and the need for cationic counter-ions leads to a high osmolality. This large osmolality difference between the complex solution and the body fluid causes very important adverse effects, such as pain and tissue sloughing when extravasated upon injection. Third, because the inner-sphere proton relaxivity is linearly proportional to the number of water molecules that are directly coordinated to the Gd³⁺ ion, it is necessary to design and synthesize novel ligands which can not only form stable complexes with the Gd³⁺ ion, but leave one more site (q=2) for a coordinated water molecule at the same time.

Based on all the considerations mentioned above, an ideal MRI contrast agent should be a neutral Gd³⁺ complex of a cyclic polyaminocarboxylic ligand that possesses at least one, or even better, two, coordinated water molecules to ensure a large relaxivity value while maintaining high thermodynamic stability and kinetic inertness. Furthermore, ideal CAs should be target-specific to highlight specific organ/tissue, or be active/inactive by consciously controlling in these areas, which will mean that the doses necessary for imaging will be reduced. The overall charge neutral Gd³⁺ complexes based on DO3A (tri-N-carboxymethyl-1,4,7,10-tetraazacyclodecane) are no doubt the best choices. In such complexes, pendant chelating moieties (including carboxylate, amides, etc.) that occupy three N-positions of the macrocycle are utilized for strong lanthanide chelation, and the remaining N-position can be derivatized freely to substantially improve the hydrophilicity, lipophilicity, tissue selectivity or other biodistribution affecting properties of the chelates, Moreover, there are two coordinated sites left for the binding of water molecules in these heptadentate complexes. For example, [Gd(HP-DO3A)(H₂O)] (ProHance™) and [Gd(DO3A-butrol)(H₂O)] (Gadovist™), both of which are based on the DO3A skeleton, have been developed and used in practice.

The tris-N alkylated 1,4,7,10-tetraazacyclododecanes also have utility in the preparation of radio-pharmaceuticals, luminescence and bio-luminescent probes, sensors, and RNA deavers.

One of the biggest drawbacks of DO3A chelates lies in their synthesis. One step selective polyalkylation of cyclen was not believed possible and all reported procedures involve a multi-step procedure. The selective functionalizations of the cyclen ligand in all reported methods are very time consuming and technically difficult. In a multi-step preparation, protection and deprotection are essential, and the target products consequently have low yields. Several routes for DO3A derivatives have been reported: (1) three amines groups in the cyclen are temporarily protected by groups such as tert-butyloxycarbonyl, tosyl and formyl, before the alkylation is performed (Kimura et al., J. Am. Chem. Soc. 1997, 119, 3068; Dischino et al., Inorg. Chem. 1991, 30, 1265; Boldrini et al., Tetrahedron Lett. 2000, 41, 6527); (2) introduction of some sterically hindered reagents including phosphoryl species, glyoxal aminal, and metal carbonyls M(CO)₆ (where M=Cr, Mo, W) in a stoichiometric ratio, which can temporarily block three of the nitrogen atoms from the inside of the tetraazamacrocycles (Filal et al., Angew. Chem. Int. Ed. Engl. 1991, 30, 560; Rohovec et al., Tetrahedron Lett. 2000, 41, 1249; Patinec et al., Tetrahedron Lett. 1995, 36, 79); and (3) direct mono N-alkylation of the cyden, after which the remaining three N positions were fuctionalized with chelating groups (Helps et al., Tetrahedron 1989, 45, 219; Li et al., Tetrahedron Lett. 2002, 43, 3217).

The most efficient and convenient method to prepare the diagnostic agents based on DO3A is selectively alkylating the three NH with chelating agents (such as the most widely used acetic acid and amides, etc.) for strong lanthanide chelating, after which various functional groups can be stoichiometrically introduced to the remaining amine in the next step. This method has recently been widely applied in the synthesis of novel diagnostic agents (Corsi et al., Chem Eur. J. 2001, 7, 64; Bruce et al., J. Am. Chem. Soc., 2000, 122, 9674). In PCT Patent WO2000-30688, tris-(tert-butoxycarbonylmethyl)-1,4,7,10-tetraazacyclododecane was coupled directly with functionalised groups to give MRI CAs for investigating the blood pool, and in Germany Patent DE 2002-10117242, two tris-(tert-butoxycarbonylmethyl)-1,4,7,10-tetraazacyclododecane molecules were linked together by halides to prepare X-ray contrast agents. However, the current methods for preparing tris-substituted cyclens is unsatisfactory because of its low yields and labour cost. For example, a tris-N alkylated cyclen was prepared from cyclen by four different protective and deprotective steps, in addition to pH adjustment in the alkylation process, in the recent work of Yoo, Reichert and Welch (Yoo et al., Chem. Comm., 2003, 766). Sammes and Parker reported the preparation of tris-N substituted cyclens through the direct alkylation between the cyclen and electrophiles, but unfortunately, the yields are around 20-40% because of the low regioselectivity (Bruce et al., J. Am. Chem. Soc., 2000, 122, 9674, Dadabhoy et al., J. Chem. Soc., Perkin Trans. 2 2002, 348). Despite the expense of the cyclen reactant, known methods use an excess of it during alkylation in order to realize a monoalkylated product. Furthermore, these procedures involve a protection, functionalization and deprotection sequence. Such multiple step routes are divergent and not always applicable. Moreover, the purification steps are usually tedious and time consuming. A more convenient and straightforward procedure to prepare CAs that are based on DO3A with high selectivity and high yield is clearly desirable.

### SUMMARY OF THE INVENTION

This invention provides a direct synthetic method to prepare tris-(tert-butoxycarbonylmethyl)-1,4,7,10-tetraazacyclododecane and a series of tris-N alkylated 1,4,7,10-tetraazacyclododecane with good selectivity in high yield. All of the starting materials and solvents are commercially available, the procedure is very easy, and all of the products can be purified by ordinary separation methods. Yields of these products are highly reproducible and the method is insensible to moisture, temperature, and the concentration of starting materials over a wide range. Single crystal X-ray analysis of tris-(tert-butoxycarbonylmethyl)-1,4,7,10-tetraazacyclo-dodecane HCl (FIG. 1) showed that the alkylated N3 on cyclen was protonated and H-bonded with the opposite N1. It appears that it is the protonation of the N3 that prohibited its further alkylation and led to the high yield of tris N-alkylated products. Other alkylating agents such as benzylbromide, allylbromide, N-2-chloroethanoyl-diphenylmethylamine, (R)-N-2-chloroethanoyl-1-6 phenylethylamine, N-2-chloroethanoyl-hexylamine and 2-bromo-propionic acid ethyl ester were also found to react with cyclen in a similar condition and gave satisfactory yields (see Table 1 below), which shows that this synthetic method could be extended to a general procedure for the preparation of tris-substituted cyclen from the reaction between the “active” alkylating agents and cyden.

TABLE 1
Yield and regioselectivity of selected electrophiles with cyclen in the
condition of CHCl₃/(Et)₃N

| Entry | Electrophiles | | Product | Yield (%)^{a} |
|---|---|---|---|---|
| 1 | 1b | 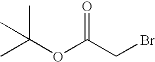 | 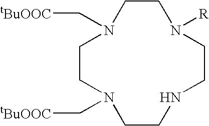 | 1. R = CH₂COOBu^{t}Tris: 77^{b}1a. R = H 1,4-Bis: 81^{C}, r > 99^{d} |
| 2 | 2b | 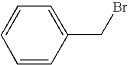 | 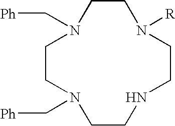 | 2. R = CH₂Ph Tris: 86%^{b}2a. R = H 1,4-Bis: 78^{C}, r > 99^{d} |
| 3 | 3b | 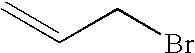 | 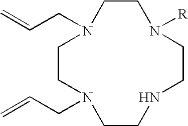 | 3. R = CH₂CH═CH₂Tris: 76%^{b}3a. R = H 1,4-Bis: 74^{C}, r > 99^{d} |
| 4 | 4b | 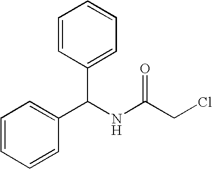 | 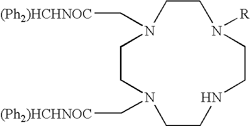 | 4. R = CH₂CONHCH(Ph)₂Tris: 81^{b}4a. R = H 1,4-Bis: 71^{C}, r > 99^{d} |
| 5 | 5b | 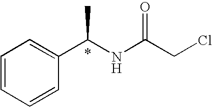 | 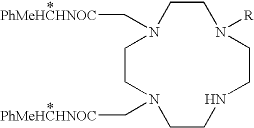 | 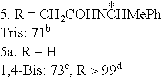 |
| 6 | 6b | 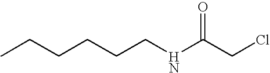 | 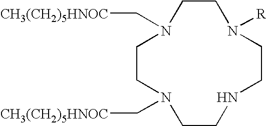 | 6. R = CH₂CONH(CH₂)₅CH₃Tris: 84^{b}6a. R = H 1,4-Bis: 75^{c}, r > 99^{d} |
| 7 | 7b | 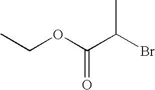 | 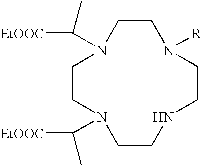 | 7. R = CHMeCOOEt Tris: 65^{b}7a. R = H 1,4-Bis: 70^{c} |

^{a}Isolated yield of purified product.
^{b}In presence of 3.5 equiv. of halids.
^{c}In presence of 2.0 equiv. of halids.
^{d}Ratio of 1,4/1,7 N-alkylated cyclen.

### DESCRIPTION OF THE INVENTION

This invention discloses the direct synthesis of tris-(tert-butoxycarbonylmethyl)-1,4,7,10-tetraazacyclododecane and a series of tris-substituted-1,4,7,10-tetraazacyclododecanes with good regioselectivity and in high yield. All starting materials, including 1,4,7,10-tetraaza-cyclododeeane (cyclen), the chosen electrophiles, solvents, and auxiliary bases are all commercially available. The procedure is easy to handle and no special reagents or harsh reaction conditions are required. In addition, the reaction is efficient; the process can be carried out within 16-20 h at room temperature. In the case of purification, simple column chromatography on aluminium oxide can yield pure tris-N alkylated products satisfactorily.

The effect of solvents on the yield of tris-(tert-butoxycarbonyl-methyl)-1,4,7,10-tetraazacyclododecane was examined and chloroform be the solvent of choice (as seen in the following Table 2).

TABLE 2
Effect of solvent and auxiliary based on yield and regioselectivity

| | | | Yield (%)^{b} | | | |
|---|---|---|---|---|---|---|
| Entry | Cond.^{a} | Base | tris | bis | tetra | r^{c} |
| 1 | CHCl₃ | Free | 51 | 40 | n.d.^{f} | >99 |
| 2 | CHCl₃ | Pyridine^{d} | 63 | 28 | n.d.^{f} | >99 |
| 3 | CHCl₃ | K₂CO₃^{e} | 35 | 32 | 27 | >99 |
| 4 | CHCl₃ | (Et)₃N^{d} | 77 | 20 | n.d.^{f} | >99 |
| 5 | CH₂Cl₂ | (Et)₃N^{d} | 62 | 32 | n.d.^{f} | >99 |
| 6 | DMF | (Et)₃N^{d} | 54 | 31 | ˜7 | 3.7 |
| 7 | CH₃CN | (Et)₃N^{d} | 48 | 25 | 21 | 2.4 |
| 8 | MeOH | (Et)₃N^{d} | 42 | 31 | 22 | 2.8 |

^{a}3.5 equiv. tert-butyl bromoacetate, 14-20 h, 298 K.
^{b}Isolated yield of purified product.
^{c}Ratio of 1,4/1,7 bis-alkylated cyclen determined by ¹H NMR and ¹³C NMR.²¹
^{d}10.0 Equiv. of (Et)₃N or pyridine.
^{e}5.0 Equiv. of K₂CO₃.
^{f}Not detect.

The use of aprotic solvents such as chloroform is preferable to polar, aprotic solvents such as dimethylformide (DMF) and polar, protic solvents such as methanol, which lead to substantial increases in tetra-(tert-butoxycaarbonylmethyl)-1,4,7,10-tetraazacyclododecane by promoting the proton transfer. In a solvent like DMF and methanol, the yield is decreased to about 40-60%.

To confirm the effect of auxiliary bases on the yields of tris-N alkylated products, comparative studies between the absence and the presence of various bases were performed in chloroform or dichloromethane (see entries 1-5, Table 2). Among the bases examined, triethylamine gave the highest yield. Switching from triethylamine to K₂CO₃ or pyridine led to a remarkable decrease in the yield, which was below 65%. It is also noteworthy that the main byproduct in this reaction condition was 1,4-bis(tert-butoxycarbonylmethyl)-1,4,7,10-tetraazacyclododecane (Li et al., J. Org. Chem. 2003, 68(7), 2956).

The applicability of the invention at larger than laboratory scale was also demonstrated. An investigation into the concentration effect of starting materials on the yields of tris-N alkylated cyclen showed that a high yield of tris-N alkylated product was also observed as the concentration of cyclen was changed from 10 to 120 mM (FIG. 2), which indicated that tris-N alkylated cyclens can be prepared on a large scale, and that this reaction can be extended to industry manufacture.

A series of experiments was conducted in the presence of tert-butyl bromoacetate from 2.0 equiv. to 8.0 equiv. to further investigate the regioselectivity and distribution of different N-alkylated products in the CHCl₃/(Et)₃N system (FIG. 3). The only two products that were isolated in the whole reaction process were 1,4-bis N-alkylated cyclen 1a and tris N-alkylated cyclen 1. The yield of 1 increased gradually and reached approximately 77% in the presence of about 3.5 equiv. of alkylating agent. At the same time, the yield of 1a decreased from 81% to 20%. Interestingly, the regioselectivity was kept nearly constant, and no tetra N-alkylated cyclen was found even in a large excess of 8.0 equiv. of electrophile.

The effects of both temperature and reaction time on the yields of the tris-N alkylated products were also investigated. The best yield was achieved when the temperature was 20-35° C. and the reaction time was 16-20 h. Tetra-N alkyated products began to emerge when the reaction temperature rose to above 60° C., probably related to the breaking of the H-band between the nitrogen atoms in the cyclen ring, and no improvement of yield was found by extending the reaction time.

### BRIEF DESCRIPTION OF THE DRAWINGS

FIG. 1 is an ORTEP drawing of tris-(tertbutoxyearbonylmethyl)-1,4,7,10-tetraazacyclododecane-1.HCl (50% ellipsoids) with selected intramolecular N - - - N distances (angstroms); N(2) - - - N(4) 3.03(1), N(1) - - - N(3) 4.60(1).

FIG. 2 plots the yield of tris-(tert-butoxycarbonylmethyl)-1,4,7,10 tetraazacyclododecane as a function of concentration of the starting material (cyclen) in CHCl₃. (298 K, 3.3 equiv. tert-butyl bromoacetate).

FIG. 3 plots yields of tris and 1,4-bis N-alkylated cyclens 1 (•) and 1a (∘) as a function of the number of the equiv. of tert-butyl bromoacetate added (298 °K, CHCl₃/(Et)₃N).

FIG. 4 is an ORTEP drawing of 1a.HCl (50% ellipsoids). Selected intramolecular N . . . N distance (Å): N(1) . . . N(3), 2.87 (1); N(2) . . . N(4), 4.82 (1).

FIG. 5 shows conformations of 12-membered cyclen ring.

FIG. 6 schematically shows the preparation of a Gd complex.

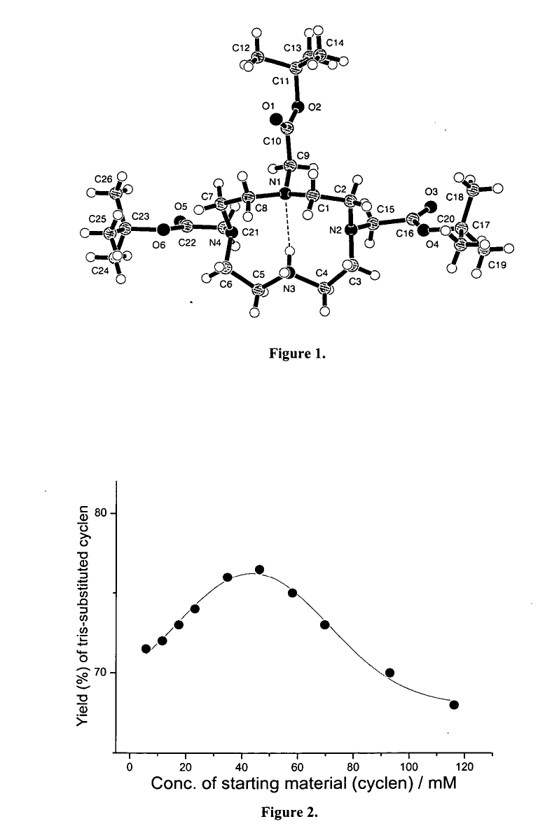

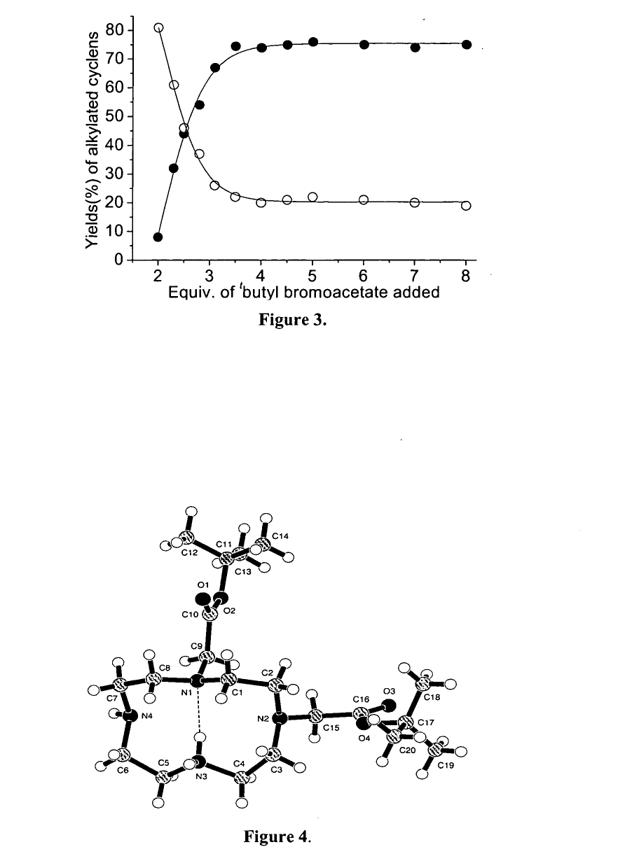

### EXAMPLES

In order to illustrate the invention, various non-limiting examples are set forth below.

### Example 1

### Tris-(tert-butoxycarbonylmethyl)-1,4,7,10-tetraazacyclododecane (1)

3.3 equivalents of tert-butyl bromoacetate (773.0 mg, 7.6 mmol) dissolved in 10.0 mL anhydrous chloroform was added dropwise to a mixture of 1,4,7,10-tetraazacyclododecane (cyclen) (400.0 mg, 2.32 mmol) and 10.0 equivalents of triethylamine (2.3 g, 23.2 mmol) in 40 mL anhydrous chloroform under an argon atmosphere for about half an hour. The reaction mixture was stirred for another 2 hours, and 0.5 equivalents of anhydrous K₂CO₃ was added. After a further 14 hours of reaction, the resulting solution was washed by water (3×40 mL). Then anhydrous Na₂SO₄ was used to dry the organic phase and the solvent was removed under vacuum to give a transparent oil. This crude product was purified by flash chromatography on aluminium oxide (dichloromethane/methane=200:5 (volume/volume), R_{f}=0.35) to give tris-(tert-butoxycarbonylmethyl)-1,4,7,10-tetraazacyclododecane (1) as a white powder (0.92 g, 1.78 mmol), yield: 77%. mp 178-180° C., ¹H NMR (400 MHz, CDCl₃): δ 3.34 (4H, s), 3.26 (2H, s), 3.05 (4H, s), 2.89-2.85 (12H, m), 1.47 (27H, s); ¹³C NMR (100 MHz, CDCl₃): δ 170.5 (2×C), 169.6 (C), 81.6 (3×C), 58.2 (3×CH₂), 51.3 (2×CH₂), 51.1 (2×CH₂) 49.2 (2×CH₂), 47.5 (2×CH₂) 28.2 (3×CH₃); ESIMS m/z 515.3 (M+H)⁺; HRFABMS m/z 515.3811 (M+H)⁺ [Calcd. For C₂₆H₅₁N₄O₆(M+H)⁺, 515.3809].

### Example 2

### Tris-[(diphenyl)methylcarbamoylmethyl]-1,4,7,10-tetraazacyclododecane (2)

3.3 equivalents of N-2-chloroethanoyl-diphenylmethylamine (1.98 g, 7.6 mmol) dissolved in 10.0 mL anhydrous chloroform was added dropwise to a mixture of 1,4,7,10-tetraazacyclododecane (cyclen) (400.0 mg, 2.32 mmol) and 10.0 equivalents of triethylamine (2.3 g, 23.2 mmol) in 40 mL anhydrous chloroform under an argon atmosphere for about half an hour. The reaction mixture was stirred for another 2 hours, and 0.5 equiv. of anhydrous K₂CO₃ was added. After a further 15 hours of reaction, the resulting solution was washed by water (3×40 mL) and the organic phase was dried by anhydrous Na₂SO₄. Removing the solvent under vacuum gave the light yellow solid. The crude product was purified by flash chromatography on aluminium oxide (dichloromethane/methane=200:10 (v/v), R_{f}=0.30) to give tris-[(diphenyl)methylcarbamoylmethyl]-1,4,7,10-tetraazacyclododecane as a colourless oil (1.58 g, 1.88 mmol), yield 81%. ¹HNMR(400 MHz, CDCl₃); δ 7.22-7.06 (30H, m), 6.18 (2H, d, J(H, H)=6.3 Hz), 5.97 (1H, d, J(H, H)=6.3 Hz), 3.27-3.17 (6H, m), 2.72-2.25 (16H, br, m); ¹³CNMR(100 MHz, CDCl₃): δ 171.5 (2×C), 171.3(C), 141.8 (4×C), 141.7 (2×C), 129.1 (12×CH), 128.1 (12×CH), 127.8 (6×CH), 59.7 (3×CH), 58.2 (3×CH₂), 51.3 (2×CH₂), 51.1 (2×CH₂), 49.2 (2×CH₂), 47.5 (2×CH₂); ESI-MS m/z 842 (M+H)⁺; HRFAB-MS/z 842.4769 (M+H)⁺ [Calcd. for C₅₃H₆₀N₇O₃ (M+H)⁺, 842.4758]. Anal. Calcd. for C₅₃H₆₀N₇O₃Cl: C, 72.46; H, 6.88; N, 11.16.-found: C, 72.25; H, 6.65; N, 11.12.

### Example 3

### Tris-[(R)-1-(1-phenyl)ethylcarbamoylmethyl]-1,4,7,10-tetraazacyclododecane (3)

3.3 equivalents of (R)-N-2-chloroethanoyl-l-phenylethylamine (1.51 g, 7.6 mmol) dissolved in 10.0 mL anhydrous chloroform was added dropwise to a mixture of 1,4,7,10 tetraazacyclododecane (cyclen) (400.0 mg, 2.32 mmol) and 10.0 equivalents of triethylamine (2.3 g, 23.2 mmol) in 40 mL anhydrous chloroform under an argon atmosphere for about half an hour. The reaction mixture was stirred for another 2 hours, and 0.5 equivalents of anhydrous K₂CO₃ was added. After a further 14 hours of reaction, the resulting solution was washed by water (3×40 mL), after which the organic phase was dried by anhydrous Na₂SO₄ and the solvent was removed under vacuum to give a white solid. The crude product was purified by flash chromatography on aluminium oxide (dichloromethane/methane=200:12 (v/v), R_{f}=0.25) to give tris-[(R)-1-(1-phenyl)ethylcarbamoylmethyl]-1,4,7,10-tetraazacyclododecane as a colourless oil (1.08 g, 1.65 mmol), yield 71%. ¹H NMR(400 MHz, CDCl₃): δ 7.39-7.04 (15H, m), 5.04-4.80 (3H, m), 3.28-3.03 (6H, br, s), 2.73-2.22 (16H, br, m), 1.45 (9H, br, s); ¹³CNMR(100 MHz, CDCl₃): δ 171.0 (C), 170.8 (C), 170.6 (C), 144.1 (C), 143.8 (2×C), 128.4 (6×CH), 127.1 (3×CH), 126.4 (6×CH), 60.7 (2×CH₂), 60.6 (CH₂), 54.2 (2×CH₂), 53.2 (2×CH₂), 52.1 (2×CH₂), 48.9 (CH), 48.7 (2×CH), 46.7 (2×CH₂), 22.5 (CH₃), 21.8 (2×CH₃); ESI-MS m/z 656 (M+H)⁺; HRFAB-MS m/z 656.4284 (M+H)⁺ Calcd. for C₃₈H₅₄N₇O₃ (M+H)⁺, 656.4288; Anal. Calcd. for C₅₃H₆₀N₇O₃Cl: C, 72.46; H, 6.88; N, 11.16-found: C, 72.25; H, 6.65; N, 11.12.

### Example 4

### Tris-(hexylcarbamoylmethyl)-1,4,7,10-tetraazacyclododecane (4)

3.3 equivalents of N-2-chloroethanoyl-hexylamine (1.36 g, 7.6 mmol) dissolved in 10.0 mL anhydrous chloroform was added dropwise to a mixture of 1,4,7,10tetraazacyclododecane (cyclen) (400.0 mg, 2.32 mmol) and 10.0 equivalents of triethylamine (2.3 g, 23.2 mmol) in 40 mL anhydrous chloroform under an argon atmosphere for about half an hour. The reaction mixture was stirred for another 2 hours, and 0.5 equivalents of anhydrous K₂CO₃ was added. After further a 12 hours of reaction, the resulting solution was washed by water (3×40 mL), after which the organic phase was dried by anhydrous Na₂SO₄ and the solvent was removed under vacuum to give a white solid. The crude product was purified by flash chromatography on aluminium oxide (dichloromethane/methane=200:9 (v/v), R_{f}=0.30) to give tris-(hexylcarbamoylmethyl)-1,4,7,10-tetraazacyclododecane 4 as a colourless oil (1.16 g, 1.95 mmol), yield 84%. ¹H NMR(400 MHz, CDCl₃); δ 7.95-7.72 (3H, br, m), 3.14-3.10 (12H, br, s), 2.75-2.63 (8H,br, m), 2.60-2.46 (8H, br, m), 1.43-1.41 (6H, br, m), 1.23-1.10 (18H, br, s), 0.77 (9H, br, s); ¹³C NMR(100 MHz, CDCl₃): δ 171.2 (C), 170.9 (2×C), 60.7 (3×CH₂), 56.2 (CH₂), 55.1 (CH₂), 53.2 (2×CH₂), 52.6 (2×CH₂), 47.1 (2×CH₂), 39.5 (CH₂), 39.4 (2×CH₂), 31.4 (2×CH₂), 31.3 (CH₂), 29.5 (2×CH₂), 29.4 (CH₂), 26.6 (2×CH₂), 26.4 (CH₂), 22.4 (3×CH₂), 13.8 (3×CH₂); ESI-MS m/z 596 (M+H)⁺; HRFAB-MS 596.5235 (M+H)⁺ [Calcd. for C₃₂H₆₆N₇O₃ (M+H)⁺, 596.5227]. Anal. Calcd. for C₃₂H₆₆N₇O₃Cl: C, 60.78; H, 10.52; N, 15.50. Found: C, 60.94; H, 10.69; N, 15.55.

### Example 5

### Tris-[ethyloxycarbonyl-1-methylmethyl]-1,4,7,10-tetraazacyclododecane (5)

3.3 equivalents of 2-bromo-propionic acid ethyl ester (1.36 g, 7.6 mmol) dissolved in 10.0 mL anhydrous chloroform was added dropwise to a mixture of 1,4,7,10-tetraazacyclododecane (cyclen) (400.0 mg, 2.32 mmol) and 10.0 equivalents of triethylamine (2.3 g, 23.2 mmol) in 40 mL anhydrous chloroform under argon atmosphere for about half an hour. The reaction mixture was stirred for another 2 hours, and 0.5 equivalents of anhydrous K₂CO₃ was added. After a further 12 hours of reaction, the resulting solution was washed by water (3×40 mL) and the organic phase was dried by anhydrous Na₂SO₄. Removing the solvent under vacuum gave a white solid. The crude product was purified by flash chromatography on aluminium oxide (dichloromethane/methane=200:14 (v/v), R_{f}=0.30) to give tris-[ethyloxycarbonyl-1-methylmethyl]-1,4,7,10-tetraazacyclododecane 5 (racemic mixture) as a colourless oil (0.71 g, 1.51 mmol), yield 65%. ¹H NMR(400 MHz, CDCl₃): δ 4.04 (6H, br, a), 3.52 (1H, m), 3.34 (2H, m), 3.03-2.36 (16H, br, m), 1.28-1.06 (18H, m); ¹³C NMR(100 MHz, CDCl₃): 8171.0 (C), 170.8 (C), 170.6 (C), 144.1 (C), 143.8 (2×C), 128.4 (6×CH), 127.1(3×CH), 126.4 (6×CH), 60.7 (2×CH₂), 60.6 (CH₂), 54.2 (2×CH₂), 53.2 (2×CH₂), 52.1 (2×CH₂), 48.9 (CH), 48.7 (2×CH), 46.7 (2×CH₂), 22.5 (CH₃), 21.8 (2×CH₃); ESI-MS m/z 473 (M+H)⁺; HRFABMS 473.3336 (M+H)⁺ [Calcd. for C₂₃H₄₅N₄O₆ (M+H)⁺, 473.3339]. Anal. Calcd. for C₂₃H₄₅N₄O₆Cl.H₂O: C, 52.41; H, 8.99; N, 10.63-found: C, 52.15; H, 8.79; N, 10.47.

### Example 6

### Tris-(benzyl)-1,4,7,10-tetraazacyclododecane.HCl (2)

3.3 equiv. of benzylbromide (1.3 g, 7.6 mmol) dissolved in 10.0 mL anhydrous chloroform was added dropwise to a mixture of 1,4,7,10-tetraazacyclododecane (cyden) (400.0 mg, 2.32 mmol) and 10.0 equiv. triethylamine (2.3 g, 23.2 mmol) in 40 mL anhydrous chloroform under argon atmosphere for about half an hour. The reaction mixture was stirred for another 2 h, and 0.5 equiv. of anhydrous K₂CO₃ was added. After a further 14 h of reaction, the resulting solution was washed by water (3×40 mL). Then anhydrous Na₂SO₄ dried the organic phase and the solvent was removed under vacuum to give a white solid. This crude product was purified by flash chromatography on aluminium oxide (dichloromethane/methane=200:9 (volume/volume), Fᵣₒ=0.40) to give tris-(benzyl)-1,4,7,10-tetraazacyclododecane.HCl 2 as a white powder (0.96 g, 2.00 mmol), yield: 86%. ¹H NMR (400 MHz, CDCl₃): δ 7.38-7.30 (8H, m), 7.28-7.22 (2H, m), 7.21-7.13 (3H, m), 6.90 (2H, d, J=6.8 Hz), 3.65 (4H, s), 3.35 (2H, s), 2.83-2.57 (16H, br, m); ¹³C NMR (100 MHz, CDCl₃): δ 138.8 (2×C), 138.1 (C), 129.6 (2×CH), 129.5 (4×CH), 128.2 (4×CH), 128.1 (2×CH), 127.6 (2×CH), 127.0 (CH), 62.2 (2×CH₂), 51.8 (CH₂), 51.2 (2×CH₂), 50.8 (2×CH₂), 50.2 (2×CH₂), 48.2 (2×CH₂); ESI-MS m/z 443 (M+H)⁺; HRFAB-MS Calcd. for C₂₉H₃₉N₄ (M+H)⁺ 443.3175, found 443.3171; Anal. Calcd. for C₂₉H₃₉N₄Cl: C, 72.70; H, 8.20; N, 11.69. Found: C, 72.56; H, 8.36; N, 11.42.

### Example 6

### Tris-(allyl-1,4,7,10-tetraazacyclododecane.HCl (3)

3.3 equiv. allyl bromide of (920.0 mg, 7.6 mmol) dissolved in 10.0 mL anhydrous chloroform was added dropwise to a mixture of 1,4,7,10-tetraazacyclododecane (cyden) (400.0 mg, 2.32 mmol) and 10.0 equiv. triethylamirne (2.3 g, 23.2 mmol) in 40 mL anhydrous chloroform under argon atmosphere for about half an hour. The reaction mixture was stirred for another 2 h, and 0.5 equiv. of anhydrous K₂CO₃ was added. After a further 14 h of reaction, the resulting solution was washed by water (3×40 mL). Then, anhydrous Na₂SO₄ dried the organic phase and the solvent was removed under vacuum to give a transparent oil. This crude product was purified by flash chromatography on aluminium oxide (dichloromethane/methane=200:12 (volume/volume), Fᵣₒ=0.31) to give tris-(allyl)-1,4,7,10-tetraazacyclododecane.HCl 3 as colorless oil (579 mg, 1.76 mmol), yield 76%. ¹H NMR (400 MHz, CDCl₃): δ 5.80-5.70 (3H, m), 5.14-5.06 (6H, m), 3.11 (6H, d, J=6.4 Hz), 2.73-2.50 (16H, br, m); ¹³C NMR (100 MHz, CDCl₃): δ 134.7 (2×CH), 130.8 (CH), 119.8 (CH₂), 118.6 (2×CH₂), 60.7 (2×CH₂), 50.3 (2×CH₂), 49.7 (2×CH₂), 49.0 (2×CH₂), 48.7 (2×CH₂), 47.9 (CH₂); ESI-MS m/z 293 (M+H)⁺; HRFAB-MS Calcd. for C₁₇H₃₃N₄ (M+H)⁺, 293.2705, found 293.2714; Anal. Calcd. for C₁₇H₃₃N₄Cl: C, 62.08; H, 10.11; N, 17.03. Found: C, 62.16; H, 10.36; N, 16.82.

### Example 7

### Tris-[ethyloxycarbonyl-1-methylmethyl]-1,4,7,10-tetraazacyclododecane (5)

3.3 equiv. of 2-bromo-propionic add ethyl ester (1.36 g, 7.6 mmol) dissolved in 10.0 mL anhydrous chloroform was added dropwise to a mixture of 1,4,7,10-tetraazacyclododecane (cyclen) (400.0 mg, 2.32 mmol) and 10.0 equiv. triethylamine (2.3 g, 23.2 mmol) in 40 mL anhydrous chloroform under argon atmosphere for about half an hour. The reaction mixture was stirred for another 2 h, and 0.5 equiv. of anhydrous K₂CO₃ was added. After a further 12 h of reaction, the resulting solution was washed by water (3×40 mL) and the organic phase was dried by anhydrous Na₂SO₄. Removing the solvent under vacuum gave a white solid. The crude product was purified by flash chromatography on aluminium oxide (dichloromethane/methane=200:14 (v/v), R_{f}=0.30) to give tris-[ethyloxycarbonyl-1-methylmethyl]-1,4,7,10-tetraazacyclododecane 5 (racemic mixture) as a colourless oil (0.71 g, 1.51 mmol), yield 65%. ¹H NMR (400 MHz, CDCl₃): δ 4.04 (6H, br, s), 3.54-3.50 (1H, m), 3.36-3.32 (2H, m), 3.03-2.36 (16H, br, m), 1.28-1.06 (18H, m); ESI-MS m/z 473 (M+H)⁺; HRFAB-MS Calcd. for C₂₃H₄₅N₄O₆ (M+H)⁺, 473.3339, found 473.3336; Anal. Calcd. for C₂₃H₄₅N₄O₆Cl.H₂O: C, 52.41; H, 8.99; N, 10.63. Found: C, 52.15; H, 8.79; N, 10.47.

### Example 8

### Preparation of Gd Contrast Agents

Tris N-alkylated 1,4,7,10-tetraazacyclododecane 1 was used to prepare the novel MRI contrast agent GdL1 efficiently in a straightforward manner and was functionalized with the guanidine group, which was introduced to promote the contrast agent's cell-permeable ability, and provide an opportunity to observe the environment inside living cells, as shown in FIG. 6.

N-benzyloxycarbonyl-2-bromoethylamine 8 was prepared by the treatment of 2-bromoethylamine hydrobromide with benzyl chloroformate in (Et)₃N/CH₂Cl₂. 8 reacted with 1 to give 9. The Cbz protected group was then removed neatly under Pd(OH)₂/C in methanol, and 10 with pendant primary amnine was obtained. N,N′-Bis(tert-butoxycarbonyl) thiourea was chosen from different guanidinylation reagents to treat with 10, and give 11 with the guanidine group. After further deprotection in TFA, the resulting ligand L1 reacted with Gd₂(CO₃)₃ to get the final complex GdL1.

N-benzyloxycarbonyl-2-bromoethylamine (8) was isolated as pale yellow oil (1.11 g, yield: 88%). ¹H NMR (400 MHz, CDCl₃): δ 7.35 (5H, m), 5.38-5.34 (1H, br), 5.09 (2H, s), 3.57-52 (2H, q, J=6.0 Hz), 3.44-3.41 (2H, t, J=5.8 Hz); ¹³C NMR (75 MHz, CDCl₃): 156.3 (C), 136.4 (CH), 128.6 (2×CH), 128.3 (CH), 128.2 (CH), 67.0 (CH₂), 42.9 (CH₂), 32.4 (CH₂); ESI-MS: m/z 258.0 [M+H]⁺; HRFAB-MS: m/z 257.0064 M⁺ [Calcd. C₁₀H₁₂O₂NBr for 257.0052]. 1-(N-Benzyloxycarbonyl ethylamine)4,7,10-tris(tert-butoxycarbonylmethyl)-1,4,7,10-tetraazacyclotetradecane (9) was isolated as a colourless viscous oil (193.8 mg, yield: 72%). ¹H NMR(400 MHz, CDCl₃): δ7.42-7.15 (5H, m), 5.04 (2H, s), 3.28-3.10 (8H, m), 2.80-2.40 (18H, m). ¹³C NMR (100 MHz, CDCl₃): δ170.9 (C), 170.8 (2×C), 156.8 (C), 137.0 (C), 128.3 (3×CH), 128.0 (2×CH), 80.7 (2×C), 80.5 (C), 66.3 (CH₂), 56.8 (CH₂), 55.8 (3×CH₂), 52.9 (2×CH₂), 52.1 (2×CH₂), 51.7 (4×CH₂), 39.5 (CH₂), 28.2 (9×CH₃); ESI-MS: m/z 692.4 [M+H]⁺; HRFAB-MS: m/z 692.4590 [M+H]⁺ [Calcd. C₃₆H₆₂O₈N₅ for 692.4598]. 1-(2-ethylamine)4,7,10-tris(tert-butoxycarbonylmethyl)-1,4,7,10-tetraazacyclotetradecane (10) was isolated as a light yellow amorphous solid (195.9 mg, yield: 81%). ¹H NMR (400 MHz, CDCl₃): δ3.21 (6H, s), 2.95-2.76 (4H, m), 2.68-2.34 (16H, m), 1.35 (27H, s); ¹³C NMR(100 MHz, CDCl₃): δ171.7 (2×C), 171.0 (C), 82.1 (2×C), 81.6 (C), 56.9 (CH₂), 56.7 (2×CH₂), 51.8 (2×CH₂), 50.7 (2×CH₂), 50.6 (2×CH₂), 50.1 (2×CH₂), 46.3 (CH₂), 37.7 (CH₂), 28.4 (9×CH₃); ESI-MS: m/z 558.3 [M+H]⁺; HRFAB-MS: m/z 558.4239 [M+H]⁺ [Calcd C₂₈H₅₆N₅O₆ for 558.4231]. 1-(2-(N,N′-Bis(tert-butylcarbonyl))guanidinyl)ethyl)4,7,10-tris(tert-butoxycarbonyl-methyl)-1,4,7,10-tetraazacyclotetradecane (11) was isolated as a colorless oil (319.0 mg, yield: 74%). ¹H NMR (400 MHz, CDCl₃): δ3.60-3.36 (2H, m), 3.28-3.18 (6H, m), 2.84-2.44 (18H, m), 1.46-1.32 (45H, m); ¹³C NMR (100 MHz, CDCl₃): δ178.9 (C), 171.1 (C), 163.5 (C), 155.9 (C), 152.7 (C), 151.3 (C), 83.0 (C), 82.5 (C), 80.6 (C), 80.5 (C), 78.9 (C), 56.5 (CH₂), 56.1 (CH₂), 56.7 (CH₂), 52.7 (2×CH₂), 52.4 (2×CH₃), 52.2 (2×CH₂), 52.0 (2×CH₂), 43.5 (CH₂), 38.7 (CH₂), 28.5 (3×CH₃), 28.4 (3×CH₃), 28.2 (3×CH₃), 28.1 (3×CH₃), 27.9 (3×CH₃); ESI-MS: m/z 800.4 [M+H]⁺; HRFAB-MS: m/z 800.5438 [M+H]⁺ [Calcd. for C₃₉H₇₄N₇O₁₀ for 800.5497]. 1-(2-Guanidinium)-ethyl-4,7,10-tris(acetic acid)-1,4,7,10-tetraazacyclotetradecane (L1) was given as a transparent glassy material (99.1 mg, yield: 92%). ¹H NMR(400 MHz, D₂O): δ 4.05-3.96 (2H, t, J=7.1 Hz), 3.78-3.66 (4H, m), 3.56-3.40 (2H, s), 3.32-2.70 (18H, m); ¹³C NMR (100 MHz, D₂O): 173.9 (C), 172.0 (C), 169.8 (C), 156.6 (C), 55.7 (2×CH₂), 53.8 (CH₂), 51.2 (2×CH₂), 50.8 (2×CH₂), 50.0 (2×CH₂), 48.8 (2×CH₂), 39.1 (CH₂), 37.3 (CH₂); ESI-MS: m/z 432.4 [M+H]⁺; HRFAB-MS: m/z 432.2596 [M+H]⁺ [Calcd. for C₁₇H₃₄N₇O₆ for 432.2571].

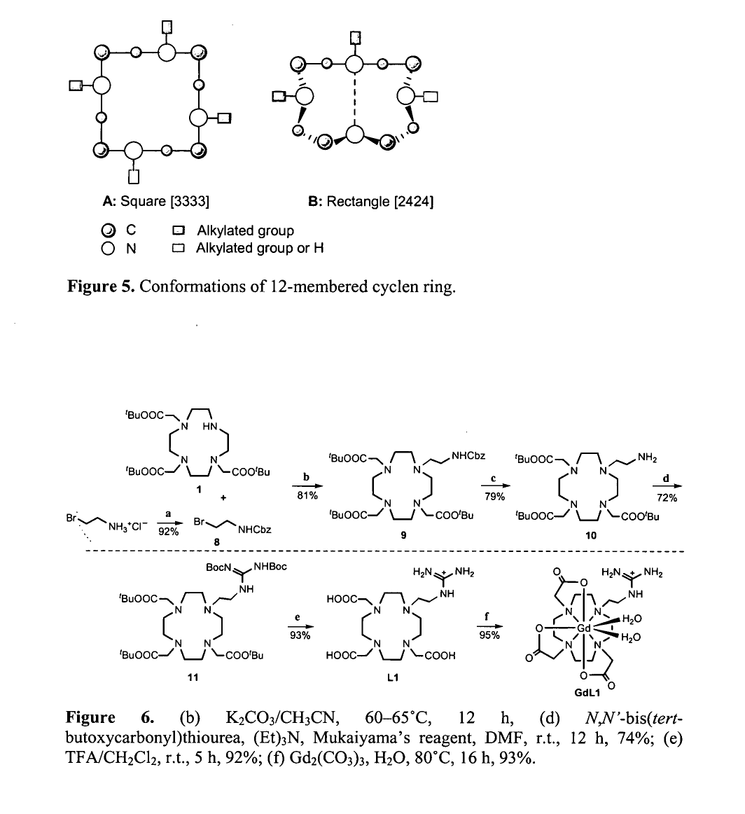

The foregoing examples show x tris N-alkylated-1,4,7,10-tetraazacyclododecanes 1-7 were synthesized through the direct reaction between cyclen and appropriate electrophiles. The products purified were characterized by ¹H NMR, ¹³C NMR, ESI-MS, and HRFAB-MS. Colorless crystals of 1 were obtained by slow evaporation of the concentrated methanol solution. X-Ray analysis revealed that the structure of 1 was in the form of its mono hydrochloride salt. In the structure of 1.HCl (FIG. 1), hydrogen bonding interaction was found between N(1) and protonated N(3), with a bonding distance of 3.032 Å, and the N—H . . . N angle was 150.2°. Without being bound by theory, it is proposed that its protonation prevents N(3) on the cyclen from being alkylated, even in the presence of a large excess of electrophiles. 1,4-Bis-(tert-butoxycarbonylmethyl)-1,4,7,10-tetraazacyclododecane is the only byproduct of significant amount in this reaction, and its yield is around 25-35% in the presence of 3.3 equiv. of alkylating agent. No tendency of these bis N-alkylated products to form higher alkylated products was found, even in the presence of excess 5.0-7.0 equiv. of electrophiles. In the structure of 1a.HCl (FIG. 4), H-bonding developed between the unalkylated N(3) and opposite N(1), with a distance of 2.867 Å, which was even shorter than that in 1.HCl, and the N—H . . . N angle was 153.5°. Furthermore, it is noteworthy that this H-bonding results in the square macrocyclar cyclen ring [3333] being “pressed” to the rectangular [2424] conformations (FIG. 5). For the two neighboring unalkylated amines in 1a, N(3) was protonated and H-bonded with the opposite N(1). Meanwhile, the nucleophilicity of N(4) decreased substantially due to its intraannular lone pair, which might explain why part of 1,4-bis N-alkylated products can not be transformed to the tris or tetra N-alkylated products even in excess of electrophiles. To promote the conversion of this 1,4-bis N-alkylated cyclen, 0.5 equiv. of anhydrous K₂CO₃, which was added in the middle of reaction process, can effectively improve the yield of tris N-alkylated cyclen. This practice was also effective for improving the yields of other tris-N substituted products under similar reaction conditions. Again without being bound by theory, it is believed that the added K₂CO₃ breaks the H-bondings that prevent the further alkylation on 1,4-bis N-alkylated cyclens. Therefore, the yields of tris N-alkylated cyclen increased.

The description above sets forth a straightforward method for the preparation of tris-substituted-1,4,7,10-tetraazacyclododecanes 1-7 in high yields, and a proposed mechanism that leads to this high regioselectivity from the stereochemical information obtained from the single crystal structure of 1 and 1a. Compared with reported work on the preparation of tris-(tert-butoxycarbonylmethyl)-1,4,7,10-tetraazacyclododecane and other tris-N-alkylated-1,4,7,10-tetraazacyclododecanes, this invention gives the highest yield and offers other attractive features such as operational convenience, cost effectiveness, and the removal of the need to use protecting groups. Furthermore, this reaction has high potential for application in practical manufacture because of the reproducible high yield in various concentrations of starting materials.

The tris-N-alkylated-1,4,7,10-tetraazacyclododecane ligands can be coordinated with a wide range of cations, such as transition metal ions and lanthanide ions, by any procedure known in the art. Some of these procedures are set forth in the art cited earlier in this description. For use in MRI, Gd complexes are preferred and these can be achieved by reacting the ligand with gadolinium oxide to form stable, neutral, water-soluble chelates.

Those skilled in the art will recognize that various changes and modifications can be made in the invention without departing from the spirit and scope thereof. The various embodiments described were for the purpose of further illustrating the invention and were not intended to limit it.

1. A method of preparing a tris-N alkylated-1,4,7,10-tetraazacyclododecane which comprises reacting 1,4,7,10-tetraazacyclododecane (cyclen) with an amount of an alkylating agent sufficient to trialkylate the cyclen in the presence of an aprotic solvent.

2. The method according to claim 1, wherein the alkylating agent is selected from the group consisting of tert-butyl bromoacetate, benzylbromide, allylbromide, N-2-chloroethanoyl-diphenylmethylamine, (R)-N-2-chloroethanoyl-1-phenylethylamine, N-2-chloroethanoyl-hexylamine, and 2-bromo-propionic acid ethyl ester.

3. The method according to claim 1, wherein the reaction is effected in a aprotic solvent.

4. The method according to claim 1, wherein the solvent is chloroform or dichloromethane.

5. The method according to claim 1, wherein the reaction is effected in the presence of a base.

6. The method according to claim 5, wherein the base comprises triethylamine, K₂CO₃, or pyridine.

7. The method according to claim 5, wherein the base comprises triethylamine and K₂CO₃.

8. The method according to claim 6, wherein the triethylamine is employed at the beginning of the reaction, and another base is added during the reaction process.

9. The method according to claim 5, wherein the base is employed at the beginning of the reaction, and another base is added during the reaction process.

10. The method according to claim 1, wherein the solvent is chloroform and the reaction is effected in the presence of triethlamine.

11. The method according to claim 10, wherein the the reaction is effected at 20 to 35° C.

12. The method according to claim 11, wherein the the reaction is effected for 16 to 20 hours.

13. The method according to claim 10, wherein the triethylamine is employed at the beginning of the reaction, and another base is added during the reaction process.

14. The method according to claim 1, wherein the the reaction is effected at 20 to 35° C.

15. The method according to claim 14, wherein the the reaction is effected for 16 to 20 hours.

16. A polyalkylated cyden compound selected from the group consisting of tris-[(diphenyl)methylcarbamoylmethyl]-1,4,7,10-tetraazacyclododecane, tris-(hexycarbamoylmethyl)-1,4,7,10-tetraazacyclododecane, and tris[ethyloxycarbonyl-1-methylrnethyl]-1,4,7,10-tetraazacyclododecane.
